(19) United States
(12) Patent Application Publication
(10) Pub. No.: US 2009/0069606 A1
(43) Pub. Date: Mar. 12, 2009

(54) Method of making alkoxylates

(75) Inventors: Zachary John Anthony Komon, Goleta, CA (US); Michael J. Weiss, Santa Barbara, CA (US)

Correspondence Address: BAKER BOTTS, LLP, 910 LOUISIANA, HOUSTON, TX, 77002-4995, US

(73) Assignee: GRT, Inc.

(21) Appl. No.: 12/215,326

(22) Filed: Jun. 25, 2008

Related U.S. Application Data
(63) Continuation of application No. 11/103,335, filed on Apr. 11, 2005, now abandoned.

(51) Int. Cl. C07C 41/09

(52) U.S. Cl. 568/619; 568/698

(57) ABSTRACT

Ethoxylates and other alkoxylates are made in an efficient manner by reacting an organic bromide with a diol in the presence of a metal oxide. An integrated process of bromide formation, alkoxylate synthesis, metal oxide regeneration, and bromine recycling is also provided.

### CROSS-REFERENCE TO RELATED APPLICATION(S)

This application is a continuation of U.S. application Ser. No. 11/103,335 filed Apr. 11, 2005, the disclosure of which is incorporated by reference herein as if set forth in its entirety.

### FIELD OF INVENTION

The invention relates generally to methods of making alkoxylates (hydroxylated ethers), and in particular relates to the synthesis of such compounds from the reaction of a brominated hydrocarbon and a diol in the presence of a metal oxide or other metal-oxygen cataloreactant. An integrated process using hydrocarbon feedstocks and metal oxide and bromine regeneration is also disclosed.

### BACKGROUND OF THE INVENTION

Alkoxylates (hydroxylated ethers), and in particular ethoxylates (e.g., mono-alkyl or aromatic ethers of ethylene glycol or ethylene glycol oligomers), are industrially significant compounds that find use as surfactants, detergents, and in other applications, either directly as the alkoxylate or after sulfation to the sulfate. The sulfated alkoxylates are superior to (non-ethoxylated) alcohol sulfates by virtue of reduced sensitivity to water hardness, less irritation to the user, and higher solubility.

Commercially important ethoxylates are typically based on hydrocarbon chain lengths of 10-18 carbon atoms, with chains as short as 6 carbon atoms and longer than 20 also used in some applications. A common measure of degree of ethoxylation is the Hydrophile-Lipophile Balance (HLB) number. The HLB number is defined as the weight percentage of ethylene oxide in the molecule divided by 5. The HLB number predicts the suitability for different applications, as shown in Table 1.

TABLE 1
HLB Values and Ethoxylate Applications

| HLB Number Range | Application |
|---|---|
| 3-6 | Water-in-oil emulsions |
| 7-9 | Wetting agents |
| 8-15 | Oil-in-water emulsions |
| 13-15 | Detergents |
| 15-18 | Solubilizers |

Another commercially important class of surfactants is based on alkyl phenol ethoxylates with chemical formula RC₆H₄(OC₂H₄)ₙOH. The most common alkyl groups, R, contain 8-12 carbon atoms and are usually branched. The desired degree of ethoxylation, n, is often 4, but ethoxylation up to n=15 is also common, and some applications may call for n as high as 70. The alkyl phenol ethoxylate-based surfactants are less common in consumer products owing to their lower biodegradability, but do find use in applications such as hospital cleaning products, textile processing, and emulsion polymerizations for which superior properties are required.

Currently, ethoxylates are produced by the addition of ethylene oxide to an alcohol. Some disadvantages to this process include: (1) the cost of ethylene oxide, (2) the volatile and unstable nature of ethylene oxide, and (3) the cost of the alcohol. The existing process also may result in a distribution in degree of ethoxylation that is not as sharp as desired. In addition to resulting in suboptimal product properties, the relatively volatile unreacted alcohol and lower ethoxylates may also negatively impact the spray drying operations used to generate the product powders.

Given the importance of alkoxylates, a new, more universal synthetic route to their production would be a welcome development. Particularly useful would be a process that uses lower cost starting materials (e.g., alkanes and ethylene glycol, rather than alcohols and ethylene oxide), avoids the use of ethylene oxide, utilizes easier (and less expensive) product purification steps, and provides more control over the degree of ethoxylation. Alcohol cost is a significant process cost and the high growth of primary alcohol ethoxylate market since the 1960s has been driven, in large part, by reductions in primary alcohol pricing. Secondary alcohols remain costly in comparison to primary alcohols, and avoiding their use by substituting alkanes will result in particularly significant improvements in process economics.

### SUMMARY OF THE INVENTION

The present invention provides methods of making alkoxylates. According to one aspect of the invention, an alkoxylate is made by allowing a brominated hydrocarbon to react with a diol in the presence of a metal-oxygen cataloreactant, preferably a metal oxide, to form an alkoxylate. For example, 2-(2′-hydroxyethoxy)-dodecane can be made by reacting 2-bromododecane with ethylene glycol in the presence of copper oxide, magnesium oxide, or other suitable metal oxide.

In a second aspect of the invention, an alkoxylate is made by forming a brominated hydrocarbon (e.g., by allowing a hydrocarbon feedstock to react with bromine), and then allowing the brominated hydrocarbon to react with a diol in the presence of a metal-oxygen cataloreactant, preferably a metal oxide, to form an alkoxylate. The invention also provides an “integrated process” in which the metal oxide and bromine are regenerated. For example, in one embodiment of the invention, dodecane is brominated to form 2-bromododecane, which is then allowed to react with ethylene glycol in the presence of a metal oxide, resulting in the formation of metal bromide(s) and alkoxylate, and the metal oxide and bromine are regenerated by allowing metal bromide(s) to react with air or oxygen.

### BRIEF DESCRIPTION OF THE DRAWINGS

These and other features and advantages of the invention will become better understood when considered in conjunction with the following detailed description, and by making reference to the appended drawings, wherein:

FIG. 1 is a schematic illustration of an integrated process for making alkoxylates according to one embodiment of the invention;

FIG. 2 is a schematic illustration of an integrated process for making alkoxylates according to another embodiment of the invention; and

FIG. 3 is a schematic illustration of a flow-type reactor for making alkoxylates according to one embodiment of the invention.

### DETAILED DESCRIPTION OF THE INVENTION

According to a first aspect of the invention, a method of making an alkoxylate is provided and comprises reacting a brominated hydrocarbon with a diol in the presence of a metal-oxygen cataloreactant, preferably a metal oxide, to form an alkoxylate. Other products (e.g., olefins, alcohols, ethers, and ketones) may also be produced. Preferably, the reaction is carried out in either the gas or liquid phase.

As used herein, an “alkoxylate” is a hydroxylated ether, i.e., an ether having at least one hydroxyl group, and includes both a hydrophobic portion and a hydrophilic portion. The alkoxylate can be aliphatic, aromatic, or mixed aliphatic-aromatic. Mixtures of alkoxylates are also included within the definition. (The term “an alkoxylate” means one or more alkoxylates.)

The term “diol” includes linear, as well as branched, dihydric alcohols. Nonlimiting examples include ethylene glycol and its oligomers (di-ethylene glycol, tri-ethylene glycol, etc.), polyethylene glycols, propylene glycol and its oligomers, polypropylene glycol, higher alkylene glycols and their oligomers, and other polyalkylene glycols.

Brominated hydrocarbons are hydrocarbons in which at least hydrogen atom has been replaced with a bromine atom, and include aliphatic, aromatic, and mixed aliphatic-aromatic compounds, optionally substituted with one or more functional groups that don't interfere with the alkoxylate formation reaction. The use of monobrominated hydrocarbons is preferred.

According to one embodiment of the invention, the reaction of a brominated hydrocarbon with a diol in the presence of a metal-oxygen cataloreactant yields an alkoxylate having the formula (1):

R′—O—(CₘH₂ₘO)ₓH (1)

wherein R¹ is alkyl (preferably C₈-C₂₀ alkyl) or R²—(C₆H₄)—, wherein R² is hydrogen, alkyl (preferably C₆-C₁₄ alkyl, more preferably C₈-C₁₂ alkyl), alkoxy, amino, alkyl amino, dialkyl amino, nitro, sulfonato, or hydroxyl; 1≦m≦4; and 1≦x≦8. It will be appreciated that —(C₆H₄)— denotes a phenylene group. In addition, where m is 2, 3, or 4, the group —(CₘH₂ₘ) can be branched or normal. Similarly, the alkyl and alkoxy group(s) can be branched or normal.

In the case where R¹ is alkyl, the alkoxylate can be represented by the formula (2):

(CₙH₂ₙ₊₁)—O—(CₘH₂ₘO)ₓH (2)

where, preferably, 8≦n≦20, 1≦m≦4, and 1≦x≦8.

In the case where R¹ is alkyl and m=2, the alkoxylate is an alkyl ethoxylate and has the formula (3):

(CₙH₂ₙ₊₁)—O—(CH₂CH₂O)ₓH (3)

where n and x are as described above. Preferred alkyl ethoxylates have an alkyl group with 8 to 20 carbon atoms, i.e., 8≦n≦20.

In the particular case where R¹ is alkyl, x=1, and m=2, the ethoxylate is a simple alkyl ether of ethylene glycol and has the formula (4):

(CₙH₂ₙ₊₁)—O—CH₂CH₂—OH (4).

Compounds having the formula (2), (3), or (4), where m=2, are mono-alkyl ethers of ethylene glycol or ethylene glycol oligomers (i.e., di-ethylene glycol, tri-ethylene glycol, etc.).

Referring again to formula (1), in the case where R¹ is R²—(C₆H₄)—, x=1, and m=2, the alkoxylate is an aromatic ethoxylate, and can be denoted by the formula (5):

R²—(C₆H₄)—O—(CH₂CH₂)—OH (5)

where R² is hydrogen, alkyl, alkoxy, amino, alkyl amino, dialkyl amino, nitro, sulfonato, or hydroxyl.

In each of formulas (1)-(5), the alkoxylate includes a hydrophobic portion (i.e., the alkyl or aromatic group) and a hydrophilic portion (i.e., the hydroxyl group and the alkoxy (CₘH₂ₘO)ₓ groups).

According to the invention, an alkoxylate is prepared by reacting a brominated hydrocarbon with a diol in the presence of a metal-oxygen cataloreactant, preferably a metal oxide. Where the alkoxylate has any of the formulas (1)-(5), the following schemes (I)-(V) can be employed:

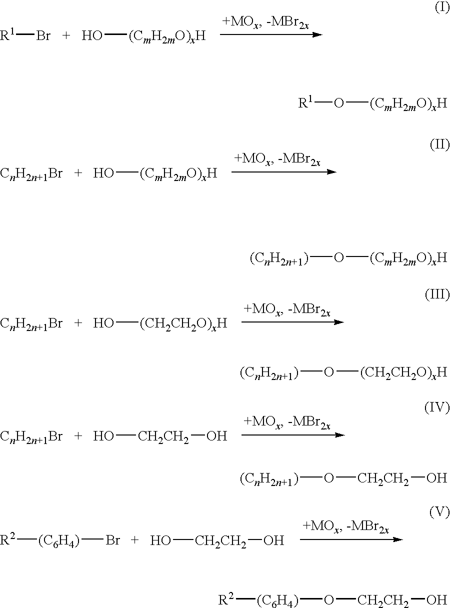

where R¹ is alkyl (preferably C₈-C₂₀ alkyl) or R²—(C₆H₄)—, where R² is hydrogen, alkyl (preferably C₆-C₁₄ alkyl, more preferably C₉-C₁₂ alkyl), alkoxy, amino, alkyl amino, dialkyl amino, nitro, sulfonato, or hydroxyl; 1≦m≦4; and 1≦x≦8. The notation “+MOₓ, -MBr₂ₓ” is not intended to denote a specific stoichiometry or empirical formula for the metal-oxygen cataloreactant, but merely refers to the interaction of the metal-oxygen cataloreactant with the reactants and the formation of metal bromides (described below).

It will be appreciated that, where x=1, the reactant HO—(CₘH₂ₘO)ₓH is an alkylene glycol, e.g., ethylene glycol (m=2), propylene glycol (m=3), and so forth. Where x>1, the reactant HO—(CₘH₂ₘO)ₓH is a di-, tri-, or polyglycol, e.g., di-ethylene glycol (x=2, m=2), tri-ethylene glycol (x=3, m=2), di-propylene glycol (x=2, m=3), and so forth. It will also be appreciated that the invention provides a convenient synthesis of a number of different alkoxylates, including mono-alkyl ethers of ethylene glycol and its oligomers, mono-alkyl ethers of propylene glycol and its oligomers, mono-alkyl ethers of other alkylene glycols and their oligomers, and aromatic ethers of various glycols and their oligomers For example, according to the invention, the reaction of a C₈-C₂₀ alkyl bromide with HO—(CₘH₂ₘO)ₓH (where m and x are as described above), in the presence of a metal-oxygen cataloreactant, results in the formation of an alkoxylate.

The diol reactant can be added to the reaction directly or, in some cases, generated in situ. For example, in one embodiment, ethylene glycol is generated in situ using 2-bromoethanol or 1,2-dibromoethane. In another embodiment, a polyol is generated in situ using a bromopropanol, dibromopropane, or other polybrominated alkane or alcohol. A combination of diols (e.g., ethylene glycol, propylene glycol, oligomers thereof, and mixtures thereof) may also be employed as reactants.

Metal-oxygen cataloreactants are inorganic compounds that (a) contain at least one metal atom and at least one oxygen atom, and (b) facilitate the production an alkoxylate. Metal oxides are representative. A nonlimiting list of metal oxides includes oxides of copper, magnesium, yttrium, nickel, cobalt, iron, calcium, vanadium, molybdenum, chromium, manganese, zinc, lanthanum, tungsten, tin, indium, bismuth, and mixtures thereof. Also included are doped metal oxides. For example, in one embodiment of the invention, any of the above-listed metal oxides is doped with an alkali metal or an alkali metal halide, preferably to contain 5-20 mol % alkali.

Particularly preferred are (i) binary oxides such as CuO, MgO, Y₂O₃, NiO, CO₂O₃, and Fe₂O₃; (ii) alkali metal-doped mixed oxides, e.g., oxides of copper, magnesium, yttrium, nickel, cobalt, or iron, doped with one or more alkali metals (e.g., Li, Na, K, Rb, Cs) (most preferably with 5-20 mol % alkali content); (iii) alkali metal bromide-doped oxides of copper, magnesium, yttrium, nickel, cobalt, or iron (alkali metal bromide dopants include LiBr, NaBr, KBr, RbBr, and CsBr); and (iv) supported versions of any of the aforementioned oxides and doped oxides. Nonlimiting examples of suitable support materials include zirconia, titania, alumina, and silica. One or more metal oxides (with or without alkali dopants) are used.

The metal oxide is perhaps best characterized as a “cataloreactant,” rather than a true catalyst, as it is converted to a metal bromide during the reaction. (For a generic metal oxide, “MOₓ,” the metal bromide(s) expected to be formed has a formula “MBr_{2x.}”) However, treating the metal bromide with oxygen or air (preferably at an elevated temperature) regenerates the metal oxide. The reaction may be generalized as MBr₂ₓ+O₂→MOₓ+Br₂, where the value of x depends on the oxidation state of the metal.

Table 2 identifies the metal bromides that are believed or predicted to be formed as a result of the metal oxide-facilitated reaction of a brominated hydrocarbon with a diol.

TABLE 2
Predicted Metal Bromides Generated from a Brominated
Hydrocarbon and Selected Metal Oxides and Dopants

| | Metal Bromide |
|---|---|
| Metal Oxide | |
| CuO | CuBr, CuBr₂ |
| MgO | MgBr₂ |
| Y₂O₃ | YBr₃ |
| NiO | NiBr₂ |
| Co₂O₃ | CoBr₂ |
| Fe₂O₃ | FeBr₂, FeBr₃ |
| CaO | CaBr₂ |
| VO | VBr₂, VBr₃ |
| MoO₂ | MoBr₂, MoBr₃, MoBr₄ |
| Cr₂O₃ | CrBr₂, CrBr₃ |
| MnO | MnBr₂ |
| ZnO | ZnBr₂ |
| La₂O₃ | LaBr₃ |
| WO₂ | WBr₂, WBr₅, WBr₆ |
| SnO | SnBr₂, SnBr₄ |
| In₂O₃ | InBr₃ |
| Bi₂O₃ | BiBr₃, BiOBr |
| Alkali Metal Dopant | |
| Li | LiBr |
| Na | NaBr |
| K | KBr |
| Rb | RbBr |
| Cs | CsBr |

Without being bound by theory, it is believed that the alkali metal in an alkali metal-doped oxide of copper, magnesium, yttrium, nickel, cobalt, or iron (and possibly others) will, upon interaction with a bromocarbon, be converted into an alkali metal bromide (LiBr, NaBr, KBr, etc.) and remain as such. It is further believed that such dopants will not provide a sink for bromine, though they will likely influence the chemistry of the metal oxide. Metal oxide supports, such as zirconia, titania, alumina, silica, etc., are not expected to be converted to their respective bromides. In an alternate embodiment of the invention, the alkoxylate product(s) and/or product distribution are altered by running the alkoxylate formation reaction in the presence of one or more ethers, alcohols, water, or other compound(s). For example, by adding tetrahydrofuran (THF), to a mixture of 2-bromododecane and ethylene glycol, the resulting product distribution is different from that obtained in the absence of THF. (Cf. Examples 5 and 6, below, (THF present) with Examples 1-4 (no THF).) Similarly, the presence of water alters the product distribution. (Cf. Example 7 (water added) with Example 1 (no water added).) A nonlimiting list of reactants that can be added to alter the alkoxylate composition/product distribution includes THF, water, and oxetane.

In a second aspect of the invention, an alkoxylate is produced in an integrated process, using a hydrocarbon feedstock. First, a hydrocarbon is brominated to generate a brominated hydrocarbon having at least one (and preferably no more than one) bromine atom. Second, the brominated hydrocarbon is reacted with a diol in the presence of a metal-oxygen cataloreactant to form an alkoxylate. One or more additional steps may also be employed. Nonlimiting examples include the separation of any undesired isomers produced in the bromination step (optionally followed by isomerization/rearrangement to yield the desired isomer, which can then be returned to the reactor and allowed to form additional product); separation of the metal bromide from the alkoxylate; and regeneration of the metal oxide and bromine using air or oxygen.

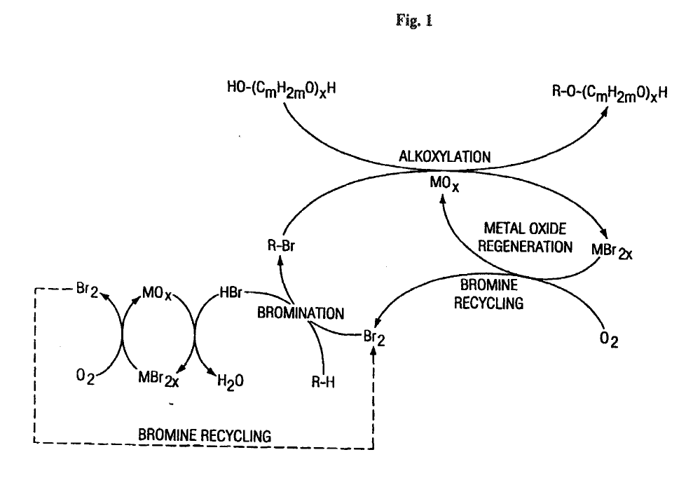

Thus, although the production of alkoxylates according to the invention can be carried out using brominated hydrocarbons purchased as commodity chemicals, it can be more advantageous to generate them as part of an integrated process that includes hydrocarbon bromination, metal-oxide-facilitated synthesis of an alkoxylate, regeneration of metal oxide, and regeneration/recycling of bromine. The process is schematically illustrated in FIG. 1. A hydrocarbon (R—H) is converted to a monobromide (R—Br), which then reacts with a glycol or glycol oligomer (HO—(CₘH₂ₘO)ₓH), where m and x are described above, in the presence of a metal oxide (MOₓ), yielding an alkoxylate and a metal bromide (MBr₂ₓ). The metal bromide is then treated with oxygen to regenerate the metal oxide and bromine.

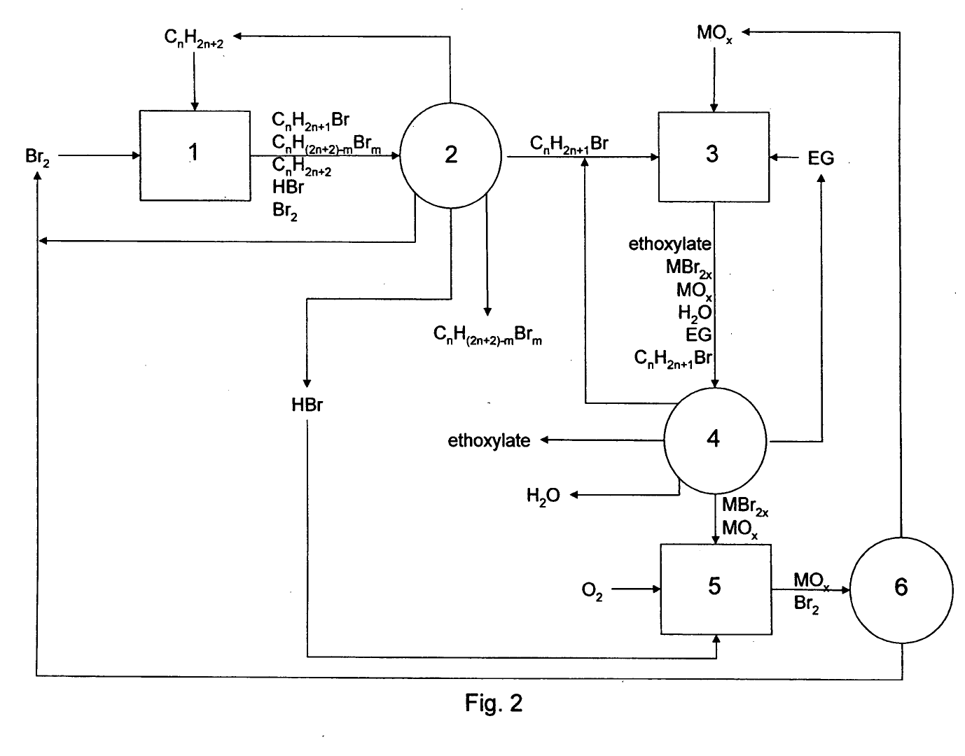

A more specific illustration of an integrated process is presented in FIG. 2, wherein ethylene glycol (EG) and an alkane are the primary reactants. In step 1, bromine (Br₂) and an alkane (CₙH₂ₙ₊₂) react to form an alkyl bromide (CₙH₂ₙ₊₂Br) and other species, which are separated in step 2. Ethoxylates are formed in step 3 by allowing the alkyl bromide to react with ethylene glycol in the presence of a metal oxide (MOₓ). The resulting ethoxylate is separated from metal bromide (MBr₂ₓ), unreacted metal oxide, and other species in step 4. The metal oxide and bromine are regenerated and recycled in steps 5 and 6.

Hydrocarbon bromination can be accomplished in a number of ways, for example, using a fixed bed reactor. The reactor may be empty or, more typically, charged with an isomerization catalyst to help generate the desired brominated isomer (see below). In an alternate embodiment, a fluidized bed or other suitable reactor is employed. A fluidized bed offers the advantage of improved heat transfer.

In one embodiment, a hydrocarbon is brominated using molecular bromine (Br₂) in the gas or liquid phase. For example, benzene can be brominated at moderate temperatures (0 to 150° C., more preferably 20 to 75° C.) and pressures (0.1 to 200 atm, more preferably 5 to 20 atm), over the course of 1 minute to 10 hours (more preferably 15 min. to 20 hrs), using FeBr₃ or another suitable catalyst. Benzene can also be brominated using FeBr₃, in the absence of Br₂, generating bromobenzene, hydrogen bromide, and FeBr₂.

In another embodiment, hydrogen bromide is used to brominate a hydrocarbon. For example, reacting an alkene with hydrogen bromide yields a bromoalkane. If the bromination reaction system carefully excludes peroxides (or, if hydroquinone or another peroxide inhibitor is added), the addition of HBr to an alkene follows Markovnikov's Rule, and the hydrogen of the acid bonds to the carbon atom in the alkene that already bears the greater number of hydrogens. Similarly, if peroxides are purposefully added to the bromination reaction, the bromination proceeds in anti-Markovnikov fashion.

Brominating an aliphatic or aromatic hydrocarbon can result in a number of different compounds, having varying degrees of bromine substitution. For example, bromination of benzene can result in the formation of bromobenzene, dibromobenzene, tribromobenzene, and more highly brominated benzene compounds. However, because the boiling points of benzene (80° C.), bromobenzene (155° C.), dibromobenzene (˜220° C.), and higher brominated isomers differ significantly, the desired isomer(s) can be readily separated from benzene and other brominated isomers via distillation. The same is generally true for other bromocarbons.

Free-radical halogenation of hydrocarbons, particularly alkanes, can be non-selective in the distribution of isomers produced. With chlorine, for example, the second chlorine is likely to attack a carbon that is non-adjacent to the first chlorinated carbon atom. (e.g., 1-chlorohexane is more likely to be chlorinated at the 3 position than at the 2 position). Although this “steering” effect is less pronounced with bromine, nevertheless, free radical bromination may give the desired isomer in some cases.

More importantly, undesired isomers can often be rearranged to more desired isomers using an isomerization catalyst, such as a metal bromide (e.g., NaBr, KBr, CuBr, NiBr₂, MgBr₂, CaBr₂, etc.), metal oxide (e.g., SiO₂, ZrO₂, Al₂O₃, etc.), or metal (Pt, Pd, Ru, Ir, Rh, and the like). In addition, various isomers often have different boiling points (up to 10-15° C. difference) and can be separated using distillation.

In some cases, the desired bromide isomer is actually the thermodynamically favored product. Thus, isomerization allows one to move from the undesirable kinetic distribution of free radical bromination to a desirable thermodynamic distribution.

Since isomerization and bromination conditions are similar, the bromination and isomerization may be accomplished in the same reactor vessel. The bromination section may be empty (no catalyst) and the isomerization section may contain the catalyst. Any dibromides or polybromides that are produced can be separated and hydrogenated to monobromides or alkane (a process referred to as “reproportionation.”)

Once the desired brominated hydrocarbon(s) is obtained, the desired alkoxylate is produced by allowing the brominated hydrocarbon(s) to react with a diol, as discussed above. The reaction can take place in any suitable reactor, including batch, semi-batch, flow, fixed bed, fluidized bed, or similar reactors, preferably made of (or lined with) glass or stainless steel. Gas phase and liquid phase reactions will now be discussed.

### Gas Phase Production of Alkoxylates

According to one embodiment of the invention, an alkoxylate is produced in the gas phase at moderate temperatures (preferably 150 to 350° C., more preferably 175 to 250° C.) and pressures (preferably 1 to 760 torr, more preferably 20 to 200 torr), in a fixed bed, fluidized bed, or other suitable reactor. Target reaction times are 0.1 seconds to 5 minutes, more preferably 1 to 10 seconds. Preferred and most preferred reaction parameters (temperature, pressure, time in reactor, etc.) can be selected based on the type and volume of the reactor, reactant and product boiling points, mole fractions, choice of metal oxide(s), and other considerations that will be apparent to a skilled person when considered in light of the present disclosure.

In one embodiment, a brominated hydrocarbon and a diol are introduced into a single, fixed bed, gas phase reactor charged with spherical or cylindrical metal oxide pellets. Alternatively, multiple reactors are employed, so that, as one is being regenerated, another is producing alkoxylates. Preferably, the metal oxide pellets have, on average, a longest dimension of 10 microns to 50 mm (more preferably 250 to 10 mm). Alternatively, the reactor is charged with comparably dimensioned spherical or cylindrical pellets of a suitable support material, such as zirconia, silica, titania, etc., onto which is supported the desired metal oxide(s) in a total amount of 1 to 50 wt. % (more preferably, 10 to 33 wt. %).

In another embodiment of the invention, products are generated in the gas phase in a fluidized bed reactor that contains metal oxide particles having, on average, a grain size of 5 to 5000 microns (more preferably 20 to 1500 microns).

For a gas phase reaction, alkoxylates are conveniently separated from metal bromide generated in the reactor by simply exhausting them from the reactor, leaving solid metal bromide behind. Optionally, saturated steam is introduced into the reactor to remove residual metal bromide (a process referred to as “steam stripping”), preferably at temperatures and pressures comparable to those used in the gas phase production of alkoxylates.

To regenerate the metal oxide in a fixed bed reactor, the bed is heated or cooled to a temperature of approximately 200 to 500° C., and air or oxygen (optionally preheated) at a pressure of 0.1 to 100 atm (more preferably, 0.5 to 10 atm) is introduced into the reactor. Bromine, and possibly nitrogen or unreacted oxygen, will then leave the bed. The bromine can be separated by condensation and/or adsorption and recycled for further use.

To regenerate the metal oxide in a fluidized bed reactor, solid metal oxide/metal bromide particles are removed from alkoxylates and any remaining reactants in a first cyclone. The particles are then fed into a second fluidized bed, heated or cooled to a temperature of approximately 200 to 500° C., and mixed with air or oxygen (optionally preheated) at a pressure of 0.1 to 100 atm (more preferably, 0.5 to 10 atm). Solid materials (regenerated metal oxide) are then separated from bromine, and possibly unreacted oxygen, in a second cyclone. The metal oxide particles can then be reintroduced into the first (or another) fluidized bed reactor. The bromine can be separated by condensation and/or adsorption and recycled for further use

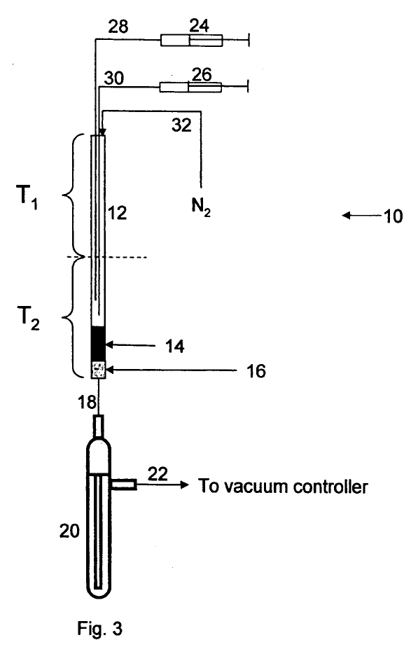

FIG. 3 illustrates one embodiment of a simple flow-type reactor for carrying out a gas phase alkoxylation. The reactor 10 includes a glass tube 12, where the alkoxylation reaction occurs. A fine powder of metal oxide 14 sits on a plug of glass wool 16 at the bottom of the glass tube. Polytetrafluoroethylene (PTFE) tubing 18 couples the glass tube to a product trap 20, which contains a liquid medium (e.g., tetradecane and octadecane). The trap is coupled to a vacuum controller (not shown) by PTFE tubing 22. Reactants are contained in separate syringe pumps 24 and 26, which are coupled to the glass reactor tube 12 by separate PTFE tubing 28 and 30. A nitrogen tank (not shown) is also coupled to the glass tube 12 by PTFE tubing 32.

After the glass tube is loaded with metal oxide, the glass tube is placed on preheated blocks (not shown). A top zone of the reactor is heated to a first temperature (T₁), and a bottom zone is heated to a higher second temperature (T₂). A nitrogen flow is started and fed into the reactor. With the product trap 20 at room temperature, the trap's pressure is lowered (e.g., to 90 torr), and reactants are fed into the reactor at a predetermined rate. After reactant delivery is complete, the glass tube is purged with nitrogen. The organic phase of the product trap is then analyzed by gas chromatography and/or or other analytical techniques.

### Liquid Phase Production of Alkoxylates

According to another aspect of the invention, an alkoxylate is produced in the liquid phase at moderate temperature (preferably 150 to 350° C., more preferably 175 to 250° C.) and pressure (preferably 0.5 to 20 atm, more preferably 1 to 7 atm), in a semi-batch, fluidized bed, or other suitable reactor. Target reaction times are 30 minutes to 24 hours (more preferably 3 to 9 hours).

In one embodiment, a simple, semi-batch reactor vessel is charged with reactants and fine metal oxide particles; alkoxylates are formed; and the products are removed. Products are separated either by increasing the reactor temperature, decreasing the reactor pressure, and/or via a solvent wash. The residual solid is regenerated in the vessel.

For a liquid phase reaction carried out in a semi-batch reactor, it is preferred to use fine metal oxide particles having, on average, a grain size of 10 microns to 5 mm (more preferably, 100 to 1000 microns).

In an alternate embodiment, alkoxylates are produced in the liquid phase in a fluidized bed, with liquid reactants, etc., flowing through a bed of fine metal oxide particles. The grain size of such particles is preferably 10 microns to 50 mm (more preferably, 250 microns to 10 mm).

For a liquid phase reaction, alkoxylates are conveniently separated from metal bromide generated in the reactor using any suitable separation technique. According to one approach, alkoxylates are vaporized (and then exhausted from the reactor) by heating the metal oxide/metal bromide/reactant/product slurry, leaving solid metal bromide behind. The metal bromide is then rinsed with a suitable organic solvent, such as octane, other alkane, or ethanol, to remove any residual alkoxylates. In one embodiment, this is carried out at 100 to 200 C, and 5 to 200 atm.

In another embodiment, alkoxylates having sufficiently low water-solubility are separated from metal bromide by exposure to water. The metal bromide dissolves, and the water-immiscible alkoxylates are separated from the aqueous metal bromide solution (e.g., gravimetrically). The bromide solution is dried, and the solid metal bromide is then regenerated. In spray drying, the metal bromide solution is sprayed into a hot zone, forming metal bromide and steam. The metal bromide particles may be separated from the steam in a cyclone prior to being regenerated with air or oxygen.

After removal of all liquids from the reactor, the metal oxide can be regenerated in a manner essentially the same as that described above for a fixed bed, gas phase reactor.

The following examples are provided as nonlimiting embodiments of the invention. In Examples 1-13, a batch reactor was used, whereas in Examples 14-19 a flow reactor of the type shown in FIG. 3 was used.

### EXAMPLE 1

A c.a. 3 mL stainless steel batch reactor was charged with 0.2549 g of electronic grade magnesium oxide (eMgO) and 0.2543 g of a 75 wt-% 2-bromododecane, 25 wt-% octadecane (as internal standard) solution. The solid and liquid were mixed by stirring with a stainless steel spatula, then 0.3065 g ethylene glycol (EG) was added. The reactor was sealed and agitated for 5 minutes with a vibratory shaker, then placed in a preheated oven at 225° C. for 6 hrs. Once cooled, the organics were extracted with ethanol and analyzed by gas chromatography as well as mass spectrometry for characterization and quantification of products and starting materials. The results of the analysis showed 49% conversion of the 2-bromododecane to products. The products consisted of 56% olefins, 3% alcohols, 40% mono-ethoxylates and 1% ketones.

### EXAMPLE 2

A c.a. 3 mL stainless steel batch reactor was charged with 0.2531 g of copper(II) oxide (CuO) and 0.2500 g of a 75 wt-% 2-bromododecane, 25 wt-% octadecane (as internal standard) solution. The solid and liquid were mixed by stirring with a stainless steel spatula, then 0.0976 g EG was added. The reactor was sealed and agitated for 5 minutes with a vibratory shaker, then placed in a preheated oven at 225° C. for 6 hrs. Once cooled, the organics were extracted with ethanol and analyzed by gas chromatography as well as mass spectrometry for characterization and quantification of products and starting materials. The results of the analysis showed 97% conversion of the 2-bromododecane to products. The products consisted of 58% olefins, 9% alcohols, 32% mono-ethoxylates and 1% ketones.

### EXAMPLE 3

A c.a. 3 mL stainless steel batch reactor was charged with 0.2501 g of copper(II) oxide (CuO) and 0.2538 g of a 75 wt-% 2-bromododecane, 25 wt-% octadecane (as internal standard) solution. The solid and liquid were mixed by stirring with a stainless steel spatula, then 0.1002 g EG was added. The reactor was sealed and agitated for 5 minutes with a vibratory shaker, then placed in a preheated oven at 225° C. for 3 hrs. Once cooled, the organics were extracted with ethanol and analyzed by gas chromatography as well as mass spectrometry for characterization and quantification of products and starting materials. The results of the analysis showed 42% conversion of the 2-bromododecane to products. The products consisted of 31% olefins, 5% alcohols, 63% mono-ethoxylates and 1% ketones.

### EXAMPLE 4

A c.a. 3 mL stainless steel batch reactor was charged with 0.2522 g of copper(II) oxide (CuO) and 0.2525 g of a 75 wt-% 2-bromododecane, 25 wt-% octadecane (as internal standard) solution. The solid and liquid were mixed by stirring with a stainless steel spatula, then 0.1001 g EG was added. The reactor was sealed and agitated for 5 minutes with a vibratory shaker, then placed in a preheated oven at 250° C. for 3 hrs. Once cooled, the organics were extracted with ethanol and analyzed by gas chromatography as well as mass spectrometry for characterization and quantification of products and starting materials. The results of the analysis showed 99% conversion of the 2-bromododecane to products. The products consisted of 58% olefins, 7% alcohols, 32% mono-ethoxylates, 1% ketones and 2% ethers.

### EXAMPLE 5

A c.a. 3 mL stainless steel batch reactor was charged with 0.2552 g of eMgO and 0.2526 g of a 75 wt-% 2-bromododecane, 25 wt-% octadecane (as internal standard) solution. The solid and liquid were mixed by stirring with a stainless steel spatula, then 0.3164 g EG and 0.6213 g of tetrahydrofuran (THF) were added. The reactor was sealed and agitated for 5 minutes with a vibratory shaker, then placed in a preheated oven at 225° C. for 6 hrs. Once cooled, the organics were extracted with ethanol and analyzed by gas chromatography as well as mass spectrometry for characterization and quantification of products and starting materials. The results of the analysis showed 88% conversion of the 2-bromododecane to products. The products consisted of 44% olefins, 4% alcohols, 48% mono-ethoxylates, 1% ketones and 3% dialkyl ethers.

### EXAMPLE 6

A c.a. 3 mL stainless steel batch reactor was charged with 0.2557 g of CuO and 0.2573 g of a 75 wt-% 2-bromododecane, 25 wt-% octadecane (as internal standard) solution. The solid and liquid were mixed by stirring with a stainless steel spatula, then 0.1320 g EG and 0.2003 g THF were added. The reactor was sealed and agitated for 5 minutes with a vibratory shaker, then placed in a preheated oven at 225° C. for 6 hrs. Once cooled, the organics were extracted with ethanol and analyzed by gas chromatography as well as mass spectrometry for characterization and quantification of products and starting materials. The results of the analysis showed 100% conversion of the 2-bromododecane to products. The products consisted of 60% olefins, 7% alcohols, 28% mono-ethoxylates, 2% ketones and 3% dialkyl ethers.

### EXAMPLE 7

A c.a. 1 ml stainless steel batch reactor was charged ¼ full of MgO, 5 drops of 75% of a 75 wt-% 2-bromododecane, 25 wt-% octadecane (as internal standard) solution, 2 drops of ethylene glycol, and 2 drops of deionized water. The reactor was sealed then placed in a preheated oven at 200° C. for 12 hrs. Once cooled, the organics were extracted with pentane and analyzed by gas chromatography as well as mass spectrometry for characterization and quantification of products and starting materials. The results of the analysis showed 92% conversion of the 2-bromododecane to products. The products consisted of 51% olefins, 36% alcohols, 11% mono-ethoxylates, 1% ketones and 1% dialkyl ethers.

### EXAMPLE 8

A c.a. 3 mL stainless steel batch reactor was charged with 0.2523 g of copper(II) oxide (CuO) and 0.2527 g of a 75 wt-% 2-bromododecane, 25 wt-% octadecane (as internal standard) solution. The solid and liquid were mixed by stirring with a stainless steel spatula, then 0.1007 g diethylene glycol (DEG) was added. The reactor was sealed and agitated for 5 minutes with a vibratory shaker, then placed in a preheated oven at 225° C. for 6 hrs. Once cooled, the organics were extracted with ethanol and analyzed by gas chromatography as well as mass spectrometry for characterization and quantification of products and starting materials. The results of the analysis showed 100% conversion of the 2-bromododecane to products. The products consisted of 42% olefins, 7% alcohols, 3% mono-ethoxylates, 46% di-ethoxylates and 2% ketones.

### EXAMPLE 9

A c.a. 3 mL stainless steel batch reactor was charged with 0.2527 g of copper(II) oxide (CuO) and 0.2491 g of a 75 wt-% 2-bromododecane, 25 wt-% octadecane (as internal standard) solution. The solid and liquid were mixed by stirring with a stainless steel spatula, then 0.1038 g diethylene glycol (DEG) was added. The reactor was sealed and agitated for 5 minutes with a vibratory shaker, then placed in a preheated oven at 225° C. for 3 hrs. Once cooled, the organics were extracted with ethanol and analyzed by gas chromatography as well as mass spectrometry for characterization and quantification of products and starting materials. The results of the analysis showed 71% conversion of the 2-bromododecane to products. The products consisted of 42% olefins, 6% alcohols, 2% mono-ethoxylates, 49% di-ethoxylates and 1% ketones.

### EXAMPLE 10

A c.a. 3 mL stainless steel batch reactor was charged with 0.2502 g of copper(II) oxide (CuO) and 0.2520 g of a 75 wt-% 2-bromododecane, 25 wt-% octadecane (as internal standard) solution. The solid and liquid were mixed by stirring with a stainless steel spatula, then 0.1056 g diethylene glycol (DEG) was added. The reactor was sealed and agitated for 5 minutes with a vibratory shaker, then placed in a preheated oven at 250° C. for 3 hrs. Once cooled, the organics were extracted with ethanol and analyzed by gas chromatography as well as mass spectrometry for characterization and quantification of products and starting materials. The results of the analysis showed 100% conversion of the 2-bromododecane to products. The products consisted of 58% olefins, 5% alcohols, 3% mono-ethoxylates, 33% di-ethoxylates and 1% ketones.

### EXAMPLE 11

A c.a. 3 mL stainless steel batch reactor was charged with 0.2516 g of copper(II) oxide (CuO) and 0.2577 g of a 75 wt-% 2-bromododecane, 25 wt-% octadecane (as internal standard) solution. The solid and liquid were mixed by stirring with a stainless steel spatula, then 0.1458 g triethylene glycol (TEG) was added. The reactor was sealed and agitated for 5 minutes with a vibratory shaker, then placed in a preheated oven at 225° C. for 6 hrs. Once cooled, the organics were extracted with ethanol and analyzed by gas chromatography as well as mass spectrometry for characterization and quantification of products and starting materials. The results of the analysis showed 95% conversion of the 2-bromododecane to products. The products consisted of 37% olefins, 5% alcohols, 1% mono-ethoxylates, 4% di-ethoxylates, 51% tri-ethoxylates and 2% ketones.

### EXAMPLE 12

A c.a. 3 mL stainless steel batch reactor was charged with 0.2498 g of copper(II) oxide (CuO) and 0.2532 g of a 75 wt-% 2-bromododecane, 25 wt-% octadecane (as internal standard) solution. The solid and liquid were mixed by stirring with a stainless steel spatula, then 0.1398 g triethylene glycol (TEG) was added. The reactor was sealed and agitated for 5 minutes with a vibratory shaker, then placed in a preheated oven at 225° C. for 3 hrs. Once cooled, the organics were extracted with ethanol and analyzed by gas chromatography as well as mass spectrometry for characterization and quantification of products and starting materials. The results of the analysis showed 80% conversion of the 2-bromododecane to products. The products consisted of 29% olefins, 6% alcohols, 1% mono-ethoxylates, 3% di-ethoxylates, 55% tri-ethoxylates and 6% ketones.

### EXAMPLE 13

A c.a. 3 mL stainless steel batch reactor was charged with 0.2516 g of copper(II) oxide (CuO) and 0.2510 g of a 75 wt-% 2-bromododecane, 25 wt-% octadecane (as internal standard) solution. The solid and liquid were mixed by stirring with a stainless steel spatula, then 0.1452 g triethylene glycol (TEG) was added. The reactor was sealed and agitated for 5 minutes with a vibratory shaker, then placed in a preheated oven at 250° C. for 3 hrs. Once cooled, the organics were extracted with ethanol and analyzed by gas chromatography as well as mass spectrometry for characterization and quantification of products and starting materials. The results of the analysis showed 100% conversion of the 2-bromododecane to products. The products consisted of 52% olefins, 5% alcohols, 2% mono-ethoxylates, 3% di-ethoxylates, 33% tri-ethoxylates, 4% ketones and 1% ethers.

### EXAMPLE 14

A flow-type reactor was assembled as shown in FIG. 3 and charged with 0.4328 g of CuO. Di-ethylene glycol (DEG) and 2-bromododecane were separately loaded into their respective syringe pumps, and c.a. 6 mL tetradecane and 207 mg octadecane were loaded into the product trap. The glass reactor tube was placed in preheated blocks to heat the top zone (T₁) to 190° C. and the bottom zone (T₂) to 200° C. A 0.4 sccm nitrogen flow was started, and the pressure in the trap was brought down to 90 torr. DEG was delivered at 500 μL/hr. After c.a. 10 minutes, 2-bromododecane was delivered at 150 μL/hr for 2 hrs. DEG delivery was continued for an additional 15 minutes, and then followed by a 15 minute nitrogen purge. The organic phase of the product trap was analyzed by gas chromatography. Analysis showed 65% conversion of the 2-bromododecane to products. The products consisted of 61% olefins, 1% alcohols, 2% mono-ethoxylates, 35% di-ethoxylates and 1% ketones.

### EXAMPLE 15

A flow-type reactor was used analogously to Example [0075]. The reactor was charged with 0.4109 g CuO. The top zone was heated to 190° C. and the bottom zone to 200° C. The product trap was charged with c.a. 6 mL tetradecane and 207 mg octadecane. The pressure was brought down to 90 torr, and DEG was delivered at 400 μL/hr. After c.a. 10 minutes, 2-bromododecane was delivered at 150 μL/hr for 2 hrs. DEG delivery was continued for an additional 15 minutes, and then followed by a 15 minute nitrogen purge. The organic phase of the product trap was analyzed by gas chromatography. The analysis showed 50% conversion of the 2-bromododecane to products. The products consisted of 59% olefins, 1% alcohols, 2% mono-ethoxylates, 38% di-ethoxylates and 1% ketones.

### EXAMPLE 16

A flow-type reactor was used analogously to Example [0075]. The reactor was charged with 0.4818 g CuO. The top zone was heated to 190° C. and the bottom zone to 200° C. The product trap was charged with c.a. 6 mL tetradecane and 208 mg octadecane. The pressure was brought down to 90 torr, and DEG was delivered at 300 μL/hr. After c.a. 10 minutes, 2-bromododecane was delivered at 150 μL/hr for 2 hrs. DEG delivery was continued for an additional 30 minutes, and then followed by a 15 minute nitrogen purge. The organic phase of the product trap was analyzed by gas chromatography. The analysis showed 70% conversion of the 2-bromododecane to products. The products consisted of 58% olefins, 2% alcohols, 2% mono-ethoxylates, 35% di-ethoxylates and 2% ketones.

### EXAMPLE 17

A flow-type reactor was used analogously to Example [0075]. The reactor was charged with 0.4328 g CuO. The top zone was heated to 190° C. and the bottom zone to 200° C. The product trap was charged with c.a. 6 mL tetradecane and 177 mg octadecane. The pressure was brought down to 90 torr, and DEG was delivered at 200 μL/hr. After c.a. 10 minutes, 2-bromododecane was delivered at 150 μL/hr for 2 hrs. DEG delivery was continued for an additional 30 minutes, and then followed by a 15 minute nitrogen purge. The organic phase of the product trap was analyzed by gas chromatography. The analysis showed 70% conversion of the 2-bromododecane to products. The products consisted of 68% olefins, 1% alcohols, 2% mono-ethoxylates, 28% di-ethoxylates and 1% ketones.

### EXAMPLE 18

A flow-type reactor was used analogously to Example [0075]. The reactor was charged with 0.4287 g CuO. The top zone was heated to 190° C. and the bottom zone to 215° C. The product trap was charged with c.a. 6 mL tetradecane and 154 mg octadecane. The pressure was brought down to 90 torr, and DEG was delivered at 300 μL/hr. After c.a. 10 minutes, 2-bromododecane was delivered at 150 μL/hr for 2 hrs. DEG delivery was continued for an additional 30 minutes, and then followed by a 15 minute nitrogen purge. The organic phase of the product trap was analyzed by gas chromatography. The analysis showed 64% conversion of the 2-bromododecane to products. The products consisted of 76% olefins, 1% alcohols, 2% mono-ethoxylates, 20% di-ethoxylates and 1% ketones.

### EXAMPLE 19

A flow-type reactor was used analogously to Example [0075]. The reactor was charged with 0.4848 g CuO. The top zone was heated to 190° C. and the bottom zone to 225° C. The product trap was charged with c.a. 6 mL tetradecane and 166 mg octadecane. The pressure was brought down to 90 torr, and DEG was delivered at 300 μL/hr. After c.a. 10 minutes, 2-bromododecane was delivered at 150 μL/hr for 2 hrs. DEG delivery was continued for an additional 30 minutes, and then followed by a 15 minute nitrogen purge. The organic phase of the product trap was analyzed by gas chromatography. The analysis showed 99% conversion of the 2-bromododecane to products. The products consisted of 89% olefins, 1% alcohols, 2% mono-ethoxylates, 7% di-ethoxylates and 1% ketones.

The present invention offers the advantages of use of lower cost starting materials (e.g., alkanes and ethylene glycol, as compared to ethylene oxide and alcohols), avoidance of ethylene oxide, use of easier and less expensive product purification steps, and more control over the degree of ethoxylation. Ethoxylation can be carried out with primary or secondary bromides. Product selectivities are similar to, and possibly higher than, that achieved with existing technology, albeit at lower conversions as compared to a hydroxylation reaction

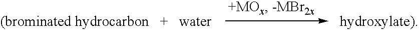

Selectivities of 40+% and 50+% for, respectively, gas-phase and liquid-phase ethoxylation, have been observed. More recently, selectivities above 85% have been observed for ethoxylation of 1-bromododecane in the liquid phase.

The invention has been described and illustrated by various preferred and exemplary embodiments, but is not limited thereto. Other modifications and variations will likely be apparent to the skilled person, upon reading this disclosure. For example, in an alternate embodiment of the invention, the reaction between a brominated hydrocarbon and a diol is carried out in the liquid phase in the absence of a metal-oxygen cataloreactant. In another embodiment of the invention, ethoxylates are produced by reacting an alkyl bromide with ethylene oxide, propylene oxide, or another organic oxide, in the presence of a metal oxide. The invention is limited only by the appended claims and their equivalents.

What is claimed is:

1. A method of making an alkoxylate, comprising:
allowing a brominated hydrocarbon to react with a diol in the presence of a metal-oxygen cataloreactant to form an alkoxylate.

2. A method as recited in claim 1, wherein the brominated hydrocarbon comprises a compound having the formula R¹—Br, where R¹ is alkyl or R²—(C₆H₄)—, where R² is hydrogen, alkyl, alkoxy, amino, alkyl amino, dialkyl amino, nitro, sulfonato, or hydroxyl.

3. A method as recited in claim 2, wherein R¹ is C₈-C₂₀ alkyl.

4. A method as recited in claim 2, wherein R¹ is R²—(C₆H₄)—, where R² is C₆-C₁₄ alkyl.

5. A method as recited in claim 1, wherein the diol comprises a compound having the formula HO—(CₘH₂ₘO)ₓH, where 1≦m≦4; and 1≦x≦8.

6. A method as recited in claim 1, wherein the diol comprises a compound having the formula HO—(CH₂CH₂O)ₓH, where 1≦x≦8.

7. A method as recited in claim 1, wherein the diol comprises ethylene glycol.

8. A method as recited in claim 1, wherein the diol comprises propylene glycol.

9. A method as recited in claim 1, wherein the diol is selected from the group consisting of ethylene glycol, propylene glycol, oligomers thereof, and mixtures thereof.

10. A method as recited in claim 1, wherein the diol is generated in situ.

11. A method as recited in claim 1, wherein the metal-oxygen cataloreactant comprises a metal oxide.

12. A method as recited in claim 11 herein the metal oxide is selected from the group consisting of oxides of copper, magnesium, yttrium, nickel, cobalt, iron, calcium, vanadium, molybdenum, chromium, manganese, zinc, lanthanum, tungsten, tin, indium, and bismuth, and mixtures thereof.

13. A method as recited in claim 11, wherein the metal oxide is selected from the group consisting of CuO, MgO, Y₂O₃, NiO, CO₂O₃, and Fe₂O₃, and mixtures thereof.

14. A method as recited in claim 11, wherein the metal oxide is doped with one or more alkali metals.

15. A method as recited in claim 11, wherein the metal oxide is alkali-doped.

16. A method as recited in claim 11, wherein the metal oxide comprises one or more alkali metal-doped mixed copper, magnesium, yttrium, nickel, cobalt, or iron oxide.

17. A method as recited in claim 16, wherein the metal oxide(s) is doped to contain 5-20 mol % alkali.

18. A method as recited in claim 11, wherein the metal oxide is doped with one or more alkali metal bromides.

19. A method as recited in claim 18, wherein the metal oxide is doped to contain 5-20 mol % alkali.

20. A method as recited in claim 11, wherein the metal oxide is supported on zirconia, titania, alumina, silica, or another suitable support material.

21. A method as recited in claim 1, further comprising including tetrahydrofuran, water, or oxetane as a co-reactant.

22. A method as recited in claim 1, wherein (a) the brominated hydrocarbon comprises a compound having the formula R¹—Br, where R¹ is alkyl or R²—(C₆H₄)—, where R² is hydrogen, alkyl, alkoxy, amino, alkyl amino, dialkyl amino, nitro, sulfonato, or hydroxyl; and (b) the diol comprises a compound having the formula HO—(CₘH₂ₘO)ₓH; 1≦m≦4; and 1≦x≦8.

23. A method of making an alkoxylate, comprising:
allowing a C₈-C₂₀ alkyl bromide to react with ethylene glycol or an ethylene glycol oligomer in the presence of a metal-oxygen cataloreactant to form an ethoxylate.

24. An integrated process for making an alkoxylate, comprising:
brominating a hydrocarbon to form a brominated hydrocarbon;
allowing the brominated hydrocarbon to react with a diol in the presence of a metal-oxygen cataloreactant to form an alkoxylate and a metal bromide; and
regenerating the metal-oxygen cataloreactant by treating the metal bromide with air or oxygen.
